(19) United States
(12) Patent Application Publication
(10) Pub. No.: US 2008/0214506 A1
(43) Pub. Date: Sep. 4, 2008

(54) Novel use of ion channel active compound, meperidine, to mediate process of accelerated wound healing

(76) Inventor: Alfred A. Nickel, Camino, CA (US)

Correspondence Address: MATHEW J. TEMMERMAN, 423 E. STREET, DAVIS, CA, 95616, US

(21) Appl. No.: 12/074,893

(22) Filed: Mar. 7, 2008

Related U.S. Application Data
(63) Continuation of application No. 10/318,940, filed on Dec. 16, 2002, now abandoned.

(51) Int. Cl. A61K 31/661; A61K 31/445; A61K 31/44; A61K 31/045; A61K 31/10; A61K 31/16; A61P 17/02

(52) U.S. Cl. 514/114; 514/317; 514/345; 514/724; 514/708; 514/625

(57) ABSTRACT

The present invention discloses a method of employing ion channel active compounds to delivery metabolism-enhancing molecules within the cell to promote rapid wound healing. It also develops a delivery kit for meperidine as a local anesthetic and wound healing accelerant for dental surgical use. And meperidine thus serves as a pattern for future design off novel wound healing compounds.

### CROSS-REFERENCE TO RELATED APPLICATIONS

This is a continuation of application Ser. No. 10/318940, filed Dec. 16, 2002, and currently pending.

### REFERENCES

U.S. Patent Documents

U.S. Pat. No. 4,847,083, Clark

U.S. Pat. No. 5,254,572, Serfontein

U.S. Pat. No. 5,395,318, Kaprelian

U.S. Pat. No. 5,612,382, Fike

U.S. Pat. No. 5,780,242 Jul. 14, 1998 Nickel A A

U.S. Pat. No. 5,776,859 Jul. 7, 1998 Nickel A A

U.S. Pat. No. 5,919,473, Elkhoury

U.S. Pat. No. 6,866,873, Stern

U.S. Pat. Pub. No. 2003/0082225, Mason

### OTHER PUBLICATIONS

1. Meperidine pkg insert. Manufactured by Baxter Healthcare Corporation (12,2001).

2. Jaffe R A, Rowe M A. “A comparison of local anesthetic effects of meperidine, fentanyl, and sufentanil on dorsal root axons”. Anesth Analg 83:776-81 (1996).

3. Hassan H G, Youssef H., Renck H. “Duration of Experimental nerve block by combinations of local anesthetic agents”. Acta Anesthesiol Scand 37:70-74 (1993).

4. Armstrong P J, Morton C P, Nimmo A F. “Pethidine has a local anesthetic action on peripheral nerves in vivo. Addition to prilocaine 0.25% for intravenous regional anaesthesia in volunteers”. Anesthesia 48(5):382-6 (5/1993).

5. Nelson K M, Vincent R G, et al. “Intraoperative intercostals nerve freezing to prevent postthoracotomy pain”. Arch Int Pharmacodyn Ther 251 (1): 150-65 (5, 1981).

6. Nickel A A. “A retrospective study of paresthesia of the dental alveolar nerves”. Anesth Prog 37:42-45 (1990).

7. Voet D, Voet J. Biochemistry John Wiley & Sons, p 727 (1995).

8. Niaspan pkg insert. Manufactured by Kos Pharmaceuticals, Inc. 400025/0600 (1999).

9. Goldstein A, Aronow L, Kalman, S. Principles of Drug Action, Harper & Rowe, p 55 (1968).

10. Mahler H R, Cordes E H. Biological Chemistry, Harper & Row, p 341 (1966).

11. Willimann H, Walde P, Luisi PL, Gazzaniga A, Stroppolo F. “Lecithin Organogel as Matrix for Transdermal Transport of Drugs.” Journal of Pharmaceutical Sciences 81(9): pp 871-874 (9/92)

### BACKGROUND OF THE INVENTION

Normal healing after surgical and third molar procedures can result in painful sequelae up to 14 days after surgery, which frequently require after-hour intervention by the surgeon. Among these sequelae are localized osteotitis (dry socket), trismus, facial edema, and protracted bleeding. For the purposes of this invention, the definition of “healed extraction site” will be a surgical wound that presents as normal clinical dental ridge architecture with pink gingival tissue and without classic granulation tissue, erythema, or edema. The present invention discloses a method that accelerates human wound healing as demonstrated in the alteration of post surgical events following exodontias and oral surgery performed by an oral and maxillofacial surgeon. The injection of a specific ion channel active compound during surgery for anesthesia management over a five year period revealed a pronounced acceleration of healing of the surgical wound over that seen in the absence of that ion channel active compound. For 15% of patients, healing occurred in 1 day; for 80% in 3-4 days; and for 5% 5-10 days. These findings have led to the novel concept of using an ion channel active chemical, an anesthetic, to act as a carrier of a desired molecular component into the ion channel in order to facilitate the entry of the molecular component into the cell itself and give it direct access to the intracellular metabolic machinery where it can act to accelerate rapid surgical wound healing.

### BRIEF SUMMARY OF THE INVENTION

Unusually rapid postoperative healing of surgical wounds has demonstrated the benefits of a method of using meperidine to function in the receptor ion channel to cause a blockade of deep bone pain and the pain controlled by local anesthetics and then to cause the desired acceleration of wound healing by virtue of intracellular metabolic stimulation.

### BRIEF DESCRIPTION OF DRAWINGS

FIG. 1 represents the ion channel accelerated wound healing process.

FIG. 2 represents design components of molecular carrier.

### DETAILED DESCRIPTION OF THE INVENTION

Meperidine, trade name Demerol, is available for medical use as an agent to support anesthesia and relief of moderate to severe pain..sup.1 Approximately six years ago, this agent was noted to clinically relieve oral surgical pain from extractions when infiltrated subcutaneously in the oral cavity buccal or labial to the extraction site. Meperidine is recognized as a deep bone receptor blockade agent and local anesthetic receptor blockade agent in both animal studies and in human obstetrical care..sup.2,3,4,5 Additionally meperidine is a central nervous system analgesic and amnesic agent.

For subcutaneous injection buccal or labial to extraction sites, meperidine is delivered in a standard insulin syringe pre-loaded with 40 mg meperidine in preservative-free solution (available as 1 cc (100 mg) ampules or 1 cc (100 mg) multidose vials). If a solution containing a preservative is used, excessive facial edema and tissue necrosis is observed clinically. Consequently, early on, the use of meperidine for oral surgery was standardized as preservative-free and limited to low doses of 5-20 mg per extraction site with a maximum of 120 mg for any one individual. Nausea is rare if the total body dose is under 120 mg. The surgical procedure also involves the concurrent use of intravenous barbituates (hexibarbitol), 0.2 mg atropine, 2 mg dexarnethasone, and concurrent use of 100% oxygen. Nitrous oxide, 20-40%, may be used intermittently during the case to assist the phobic dental patient through the procedure. Additionally each patient is encouraged to take orally 2000 units of vitamin C (adult dose) the day before surgery and 2000 units per day for 5 days postoperatively. The intravenous and inspired gas agents comprise a standard regime that has been used consistently for 28 years in this private oral surgery practice, and this method of anesthetic management was published in 1993 in articles focusing on paresthesia and post-operative sequelae..sup.6 In these 28 years, the only change in this method of patient anesthetic care has been, since March 1997, the elimination of aminobenzene local anesthetics and the substitution of meperidine as the “local” by subcutaneous infiltrating injection adjacent to and in the immediate vicinity of the surgical site. (A thiophene local anesthetic has been used on rare occasions.)

The insulin syringe has a short needle (28 gauge.times.½ inch), preventing its use to achieve the traditional dental nerve block and thus also preventing the development of post-operative paresthesia which has been noted to occur in nerve block techniques with meperidine. The infiltration process usually includes 5-15 mg in the dental sulcus with 2-3 mg infused in the attached gingiva. No periodontal ligament or intraosseous injection of meperidine is used.

Immediate postoperative pain management is unique using meperidine infiltration. All conscious patients are fully functional in the oral cavity. They can immediately talk clearly, drink fluids, and eat because the “numbness” resulting from the use of traditional local anesthetics is not present. Meperidine is a pain ion channel blockade agent without any marked clinical numbness. However, paresthesia can occur demonstrating that meperidine acts and can get “stuck” in the ion channel. Some patients on regaining consciousness require oral narcotic management, some oral ibuprofen management, some aspirin, some require nothing at all. Each surgical patient is telephoned the evening of surgery to monitor progress and many of them report at this time that they have not required the post-operative prescribed narcotics because they are not experiencing pain.

Patients are sutured for management of bleeding, but with meperidine something unusual occurs during the surgery. With four impacted third molars, for example, prior to March 1997, expected blood loss was 70-150 cc per case. After March 1997, when meperidine was used as the local anesthetic instead of an aminobenzene, blood loss per case dropped to 2-10 cc routinely and rarely went over 20 cc. Since the surgical area is not flooded with blood, the surgeon is able to dearly visualize the bony exodontia sites. Additionally, within 2-5 minutes, an extraction socket will have formed a complete clot with no further bleeding from that location during the case. This rapid clotting during surgery is seen three minutes after injection of meperidine. (If a patient has a pre-existing bleeding disorder, early clotting is not seen; but in the case of a patient with a factor deficiency disease, clotting was satisfactory for discharge 15 minutes after suturing.) Initially this lack of intraoperative bleeding was thought to be due to the elimination of the vasodilating aminobenzene and thiophene local anesthetics, however further observation of this effect suggests that it is more probably the result of the increase in intracellular metabolic rates.sup.7 caused by the metabolites of meperidine produced by the ion channel.

Postoperative pain management over 5 years has been greatly simplified since the introduction of meperidine as the local anesthetic because “dry sockets” have disappeared from the practice. Before the dry socket can occur on day 5 postoperatively, 80% of patients have healed. Even as early as twenty-four hours after surgery, some patients have presented with a dental alveolar ridge that is pink with no evidence of an extraction socket and no tissue edema or redness suggestive of a surgical procedure. A majority of cases (60-80%) present 3-4 days post surgery with pink surgical site tissue including the mucoperiostial flap. These pink, non-erythemic wound sites clinically appear similar to patients having treatment in a hyperbaric oxygen chamber, but no hyperbaric care has been given. Meperidine then acts to promote metabolic rates favorable to rapid tissue repair. In these 5 years there have been no post-operative infections and no cases of trismus or temeromandibular joint dysfunction requiring care with physical therapy. Intraoperatively cardiac monitored arrythmias have disappeared..sup.8 Laryngiospasms requiring the use of muscle paralyzing agents are not experienced.

This rapid wound healing process was noted initially in the Walnut Creek, Calif. surgical practice in March 1997. In April 2001, the surgeon relocated his home more than 120 miles east of Walnut Creek. For 10 months, through February 2002, based on the observed rapid clotting and wound healing noted with meperidine, the surgeon drove 2.5 hours in the morning to Walnut Creek, did 3-4 surgical procedures, and then drove 2.5 hours home the same day. No post-operative bleeding or wound management problems developed even though prophylactic antibiotics were not always used. At the new practice location in El Dorado Hills, Calif., (1 hour from home), after 7 months, no post-operative wound management problems have required a return to the office. In fact, the beeper service since March 1997 for after hour patient needs has been silent. Staff and patient population both in the El Dorado Hills practice have become acutely aware of the rapid post-operative healing as seen in the Walnut Creek location.

As described in U.S. Pat. Nos. 5,780,242 and 5,776,859, the molecular ion channel serves as a receptor that can also include a site for enzyme hydrolysis. The meperidine moleculeqhas an affinity to the ion channel site..sup.9 Once inside the channel, it will immediately undergo hydrolysis of its esterfied alcohol bond in the same way as other local anesthetics do. The meperidine molecule then has the head (ring structure) and tail (esterized alcohol) structure that characterizes known aminobenzene and thiophene local anesthetics.

The residual ring structure has the potential to reduce to pyridoxal phosphate analogues (Vitamin B-6 analogues) at an intracellular level of activity. “Pyridoxal phosphate is the most striking of the known coenzymes in terms of the multiplicity of different enzymatic reactions that are dependent on its presence.”.sup.10 By coenzyme function Vitamin B-6 analogues inpact alpha-amino acid degradation to high metabolic fuels. “Alpha-amino acids, in addition to their role as monomeric units, are energy metabolites and precursors of many biologically important nitrogen-containing compounds, notably heme, physiologically active amines, glutathione, nucleotides, and nucleotide coenzymes . . . amino acids are also precursors of glucose, fatty acids, and ketone bodies, and are therefore metabolic fuels.”.sup.7 Thus sudden high intracellular levels of Vitamin B-6 analogues would dictate rapid cellular metabolic rates via coenzyme activity. Concurrent usage of oxygen during the surgical procedure would then provide an environment favorable to a rapid wound repair mechanism mediated by Vitamin B-6 analogues via known coenzyme pathways similar to those induced by hyperbaric oxygen but brought about through a biochemical hypermetabolic state instead. The Vitamin B-6 analogues can elevate action potential thresholds to reduce transmission of neuron pain impulses. The desirable phenomena of clinically observable rapid wound healing, rapid blood clotting, lack of cardiac arrythmias, and minimization of post-operative pain are explained by the production of ion channel metabolites from meperidine.

The meperidine molecule can thus first impede ion channel function with its local anesthetic component; and then, through the coenzyme activity of its intracellular molecular component produced in its metabolism in the ion channel, it can cause rapid surgical wound healing. Further analysis of these actions of meperidine would allow the design of new molecules which, having an affinity to the ion channel in the cell wall, could introduce agents to promote wound healing directly into cells at the site of injury. Once present in the wall, the molecule would be metabolized to its pre-designed molecular component (Vitamin B analog) capable of targeting rapid intracellular metabolism and cell repair processes without causing changes in the DNA that might negatively alter cellular architecture.

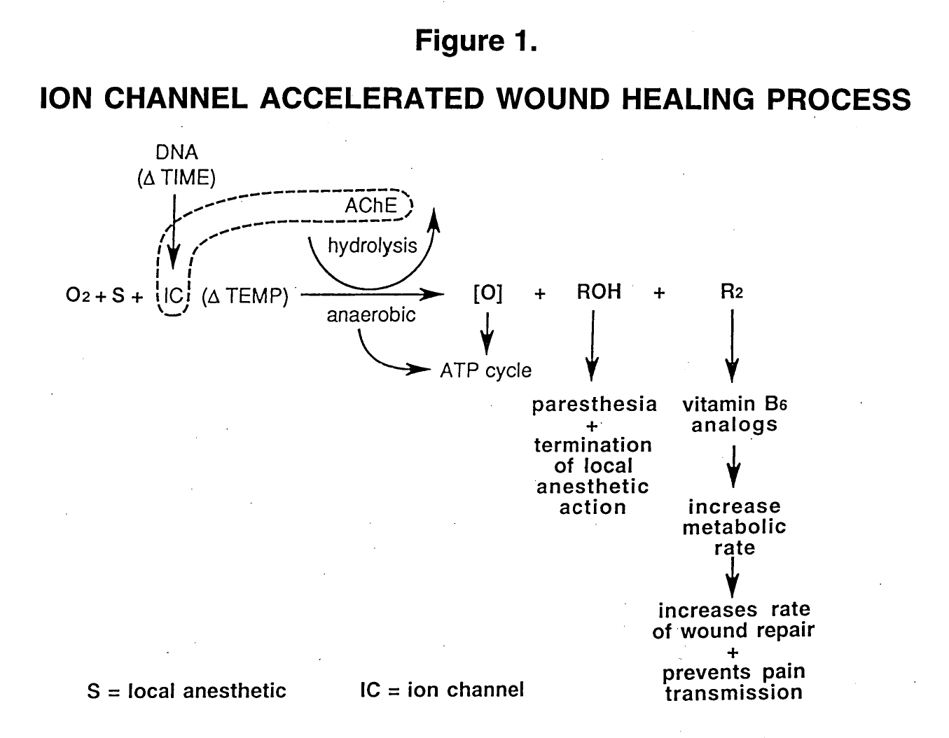

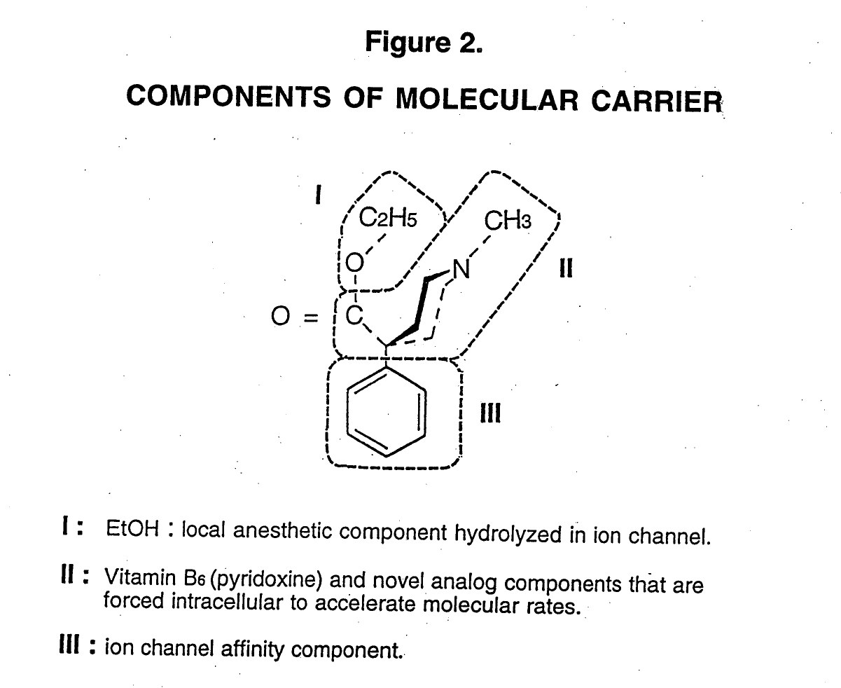

Referring to FIGS. 1 and 2, another product that has activity in humans or animals to reduce swelling, hematoma, and post-surgical pain consists of Vitamin B-6 powder suspended in a transdermal transport cream. An example of such a transdermal cream would be soy lecithin in isopropyl palmitate 50/50, pluronic F127 (poloxamer 407 NF) 20% in water, B-5 powder 5%, B-6 powder 30%. “Applying a small amount of this cream to the outside of a patient's oral surgical site 4 times daily reduces facial edema in 36-48 hours instead of 5-8 days.

Indurated hematoma present in the edema can see resolution in 14-18 hours rather than 5-8 days. Finally, patients report lack of postoperative pain compatible with the theory represented in FIG. 1 such that the psychological remorse normally resulting from the experience of chronic moderate surgical pain is gone.

Another example of this cream's ability to accelerate healing is demonstrated in its use after a hammer blow to the human thumb which produced subdermal “blood” blister, pulsating pain, and nail damage.

Application of a transdermal cream containing B-5 (5%) and B-6 (30%) after the injury resulted in compete cessation of pain in 8 minutes, blood blister hematoma completely resolved in 12-14 hours, return of function and normal tissue contours in 48 hours. Normally such an injury to the thumb from a hammer blow may take up to 3 months to heal to the degree seen in 48 hours after use of the B-5, B-6 transdermal cream. Thus analysis of FIGS. 1 and 2 gives a novel method of vitamin delivery to human tissue that results in clear cut accelerated wound healing.

1.) A method of accelerating wound healing for a patient, the method comprising:
applying to a patient a medicament for transdermal administration, oral administration, or ophthalmological administration, the medicament comprising EtOH, vitamin B6 or analog thereof, and an ion channel affinity component;
providing a transport vehicle of meperidine, lecithin, or DMSO for transfer of said vitamin B6 or analog thereof through a cell membrane in said patient's cells for the purpose of accelerating wound healing, wherein said transfer is facilitated by said ion channel affinity component acting on a cell ion channel.

2.) The method of claim 1 wherein said ion channel affinity component is used in combination with said transport vehicle.

3.) The method of claim 2 wherein said ion channel affinity component acts on an ion channel and wherein said EtOH comprises a local anesthetic component hydrolyzed in said ion channel.

4.) The method of claim 3 wherein said vitamin B6 or analogue thereof is present in an amount approximately 30% by weight.

5.) The method of claim 1 wherein said EtOH comprises a local anesthetic component hydrolyzed in said cell ion channel.

6.) The method of claim 5 wherein said-medicament further comprises at least 5% by weight vitamin B5.

7.) The method of claim 1 wherein said accelerated wound healing occurs through an increase in cellular metabolism.

8.) The method of claim 7 wherein said ion channel affinity component is used in combination with said transport vehicle.

9.) The method of claim 8 wherein said transport vehicle is meperidine and wherein said meperidine impedes the function of said ion channel.

10.) The method of claim 9 wherein said vitamin B6, or analogue thereof is present in an amount approximately 30% by weight.

11.) The method of claim 10 wherein said EtOH comprises a local anesthetic component hydrolyzed in said cell ion channel.

12.) The method of claim 1 further wherein said transport vehicle is meperidine and wherein said meperidine impedes the function of said cell ion channel.

13.) The method of claim 12 wherein said accelerated wound healing occurs through an increase in cellular metabolism.

14.) The method according to claim 13 wherein said medicament is a topical cream.

15.) The method according to claim 14 wherein said medicament is injected.

16.) A method comprising the use of vitamin B6 or an analogue thereof in combination with a transport medium, said medium being meperidine, lecithin or DMSO for the manufacture of a local anesthetic.
